(19) United States
(12) Patent Application Publication
(10) Pub. No.: US 2006/0122613 A1
(43) Pub. Date: Jun. 8, 2006

(54) Fixator for a fractured bone

(76) Inventor: John M. Kirsch, Stevens Point, WI (US)

Correspondence Address: Jeffrey S. Sokol;ANDRUS, SCEALES, STARKE & SAWALL, LLP, Suite 1100, 100 East Wisconsin Avenue, Milwaukee, WI, 53202-4178, US

(21) Appl. No.: 11/001,828

(22) Filed: Dec. 2, 2004

(51) Int. Cl. A61B 17/56

(52) U.S. Cl. 606/74

(57) ABSTRACT

The present invention relates to a fixator for aligning and setting a fractured bone with an internal canal, such as a Colles' fracture of the radius. The fixator includes two positioning wires and at least one pinball fastener. An elongated portion of each wire is inserted through either the radial styloid process or the dorso-ulnar cortex and into the intramedullary canal. A shaped portion of each wire extends from the wrist. The lateral portion of the shaped portions are coplanar when viewed from above and their overlapping portions are in side-by-side relation when viewed from the side. The external portions are also preferably curved with concentric overlapping portions so that the fixator resembles a bracelet. When necessary, the fixator includes additional interfragmentary stabilizing wires that are securely joined to the positioning wires. Crimp sleeves can be used in conjunction with the pinballs to securely join the wires.

### BACKGROUND OF THE INVENTION

One of the most common fractures caused by falling on the palm of the hand 5 is a break in the lower third of the radius bone 10 as shown in FIG. 1. This fracture 20 is commonly referred to as a Colles' fracture. The distal end 11 of the radius 10 joins the hand bones at the wrist 4. The distal end 11 has a generally concave, or partial cup shaped surface. When viewed from above as in FIG. 2, the surface forming the distal end 11 is generally angled about 15 degrees (15°) out of normal from a longitudinal axis 14 of the radius 10. The thumb side extends further forward. Similarly, the top and bottom portions extend further forward than the central portion. This cup-shaped surface is aligned with the longitudinal axis 14 to face forward toward the wrist 4. This structure helps give the wrist 4 its full range of mobility and added strength. When the distal end 11 is broken, proper alignment and setting of the pieces 21 and 22 of bone 10 is important to return the wrist 4 to full mobility and strength. If the piece 21 containing the distal end 11 reattaches to the main portion of bone 22 out of alignment with the longitudinal axis 14, the bone structure, tendons and ligaments will inhibit full mobility and strength even after months of physical therapy.

Setting and maintaining proper alignment of the pieces 21 and 22 of bone 10 in a Colles fracture 20 can be difficult. The distal piece 21 is easily dislodged due to the ongoing use of the hand and arm. When the fracture 20 is angled out of normal to the longitudinal axis 14 of the bone 10, the pieces slide out of alignment even more easily. Although the forearm 3, wrist 4 and hand 5 can be placed in a cast, this prolonged immobilization of the wrist 4 typically requires many weeks or months of expensive physical therapy, and the wrist may not return to full mobility and strength.

A conventional fixator developed for setting a Colles' fracture without casting is shown and described in U.S. Pat. No. 5,571,103. The fixator has two wires that are inserted through its distal end at the radial styloid process and dorso-ulnar cortex and extend through the intramedullary canal of the radius. The wires also extend through the skin to a location above the wrist where they are joined by a clamp. A problem with this fixator is that the wires are spaced apart at the clamp. Although attempts are made to prevent rotation of the wires, the constant use of the arm, wrist and hand can alter the alignment of the pieces and of bone. The fixator is prone to frequent bumps and jostles.

Another problem with the conventional fixator is its bulky and awkward size, weight and unattractive appearance. The patient is continually reminded of the presence of the fixator each time he or she moves his or her arm due to its size and weight. The boxy shape of the clamp and wires are prone to bumps that do not glance off and impart significant force. The projecting free ends of the wires can be easily snagged and pulled to cause pain and misalignment of the fixator. The large bulky clamp and wires are also a constant visual reminder that can cause unwanted attention from others and emotional uneasiness to the patient, particularly at professional or formal engagements where the patient cannot easily conceal the fixator without attracting attention.

A further problem with the conventional fixator is its relatively high cost. The clamp is not a standard fastener. Replacing the clamp is difficult should it break or malfunction, such as via its screw stripping due to over tightening. Maintaining an inventory of clampsis expensive.

The present invention is intended to solve these and other problems.

### BRIEF DESCRIPTION OF THE INVENTION

The present invention relates to a fixator for aligning and setting a fractured bone with an internal canal, such as a Colles' fracture of the radius. The fixator includes two positioning wires and at least one pinball fastener. An elongated portion of each wire is inserted through either the radial styloid process or the dorso-ulnar cortex and into the intramedullary canal. A shaped portion of each wire extends from the wrist. The lateral portion of the shaped portions are coplanar when viewed from above and their overlapping portions are in side-by-side relation when viewed from the side. The external portions are also preferably curved with concentric overlapping portions so that the fixator resembles a bracelet. When necessary, the fixator includes additional interfragmentary stabilizing wires that are securely joined to the positioning wires. Crimp sleeves can be used in conjunction with the pinballs to securely join the wires.

One advantage of the present fixator invention is its ability to maintain the broken pieces of bone in alignment. The external portions of the positioning wires are angled 15° out of normal and their overlapping portions are in side-by-side relation. The overlapping portions of the wires are not spaced apart. Any inadvertent rotation of one overlapping portion about the other does not significantly displace either wire. This side-by-side or flush mating arrangement helps maintain the position of the wires in the intramedullary canal and the alignment of the pieces 21 and 22 of broken bone.

Another advantage of the present fixator invention is its light weight, streamline shape and relatively attractive appearance. The light weight and streamline design of the fixator is less noticeable to the patient when he or she moves his or her arm. The curved, coplanar and concentric shape of the wires, and the small round pinballs and crimp sleeves minimize bumping against other objects. When bumps do occur they tend to glance off and do not impart significant force. The free ends of the wires are easily concealed by pinballs so that they do not snag and pull on foreign objects. The streamlined wires and colored pinballs produce a more attractive appearance that reduces the amount of attention the fixator draws. Although broken bones and fixators are rarely desired fashion accessories, this fixator and its colored pinballs does bear a resemblance to a bracelet to reduce the emotional unease of the patient.

A further advantage of the present fixator invention is that it is provided as a kit. Each kit contains all the components a surgeon needs to assemble the fixator. The kit can be used to make a relatively simple fixator with only two positioning wires and a single pinball, or a fixator having additional stabilizing wires, pinballs or crimp sleeves. The use of inexpensive pinball and crimp sleeve fasteners enables a single kit to include a variety of these fasteners so that the surgeon and patent can pick and choose the desired configuration and color of the fixator. The hospital inventory need only stock a few of the kits.

A still further advantage of the present fixator invention is its one size fits all design. The fixator is designed to fit a very wide array of body sizes, shapes and ages. The clockwise rotation of the external portion of one wire and the counter-clockwise rotation of the external portion of the other wire enable the fixator to increase or reduce its effective diameter or size so that it can be adjusted to fit around a wide variety of wrist shapes and sizes.

A still further advantage of the present fixator invention is its use of readily available and relatively inexpensive fasteners. The pinballs and crimp sleeves are standard, commercially available fasteners that are relatively inexpensive and stock items in most operating rooms. Replacing these fasteners is easy. Extras parts are included in each kit or can be stocked in abundance at relatively no cost. The inventory and ordering of kits is simplified because the inexpensive pinballs enable each kit to contain a variety of pinball colors.

Other aspects and advantages of the invention will become apparent upon making reference to the specification, claims and drawings.

### BRIEF DESCRIPTION OF THE DRAWINGS

FIG. 1 is a perspective view showing a first embodiment of the fixator surgically placed with the elongated longitudinal portion of each positioning wire inserted into the intramedullary canal and through a fracture of the radius to align its broken pieces, and with the lateral or shaped portion of each positioning wire protruding from and extending across the wrist of the individual where their overlapping portions are joined together by a pinball.

FIG. 2 is a top view of FIG. 1 showing the surgically placed fixator in the fractured radius.

FIG. 3 is an end view of FIG. 1 showing the entry point of each longitudinal portion in the distal end of the radius and the continuously curved shape of each lateral portion with their concentrically aligned overlapping portions joined by the single pinball.

FIG. 4 is a top view of the radial styloid process (RSP) wire for the first embodiment in its manufactured, pre-surgical placement configuration.

FIG. 4A is a partial front end view of the RSP wire of FIG. 4 showing the continuously curvature of its lateral portion and its high curvature and low curvature portions.

FIG. 5 is a side view of the dorso-ulnar cortex (DUC) wire for the first embodiment in its manufactured, pre-surgical placement configuration.

FIG. 5A is a partial front end view of the DUC wire of FIG. 5 showing the low curvature shape of its lateral portion.

FIG. 6 is a top view of the surgically placed fixator with positioning wires having continuous curve securement ends joined by two pinballs and a crimp sleeve, and with two cross wires joined to the positioning wires by crimp sleeves to further stabilize the fracture.

FIG. 7 is an end view of FIG. 6 showing the surgically placed fixator.

FIG. 8 is a side view of FIG. 6 showing the surgically placed fixator.

FIG. 9 is a bottom view of FIG. 6 showing the surgically placed fixator.

FIG. 10 is a top view of a second embodiment of the fixator with positioning wires having angled lateral portions with linear segments and with their overlapping portions joined by two pinballs that straddle a central crimp sleeve.

FIG. 11 is an end view of FIG. 10 showing the surgically placed fixator with its angled lateral portions and their linear segments.

### DESCRIPTION OF THE PREFERRED EMBODIMENTS

While this invention may be embodied in many different forms, the drawings show and the specification describes in detail several preferred embodiments of the invention. It should be understood that the drawings and specification are to be considered exemplifications of the principles of the invention. They are not intended to limit the broad aspects of the invention to the embodiments illustrated.

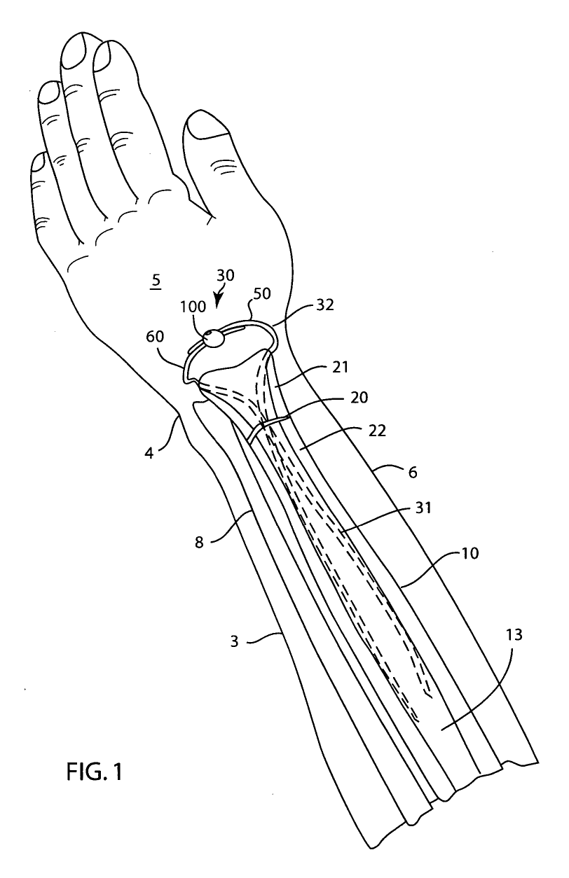

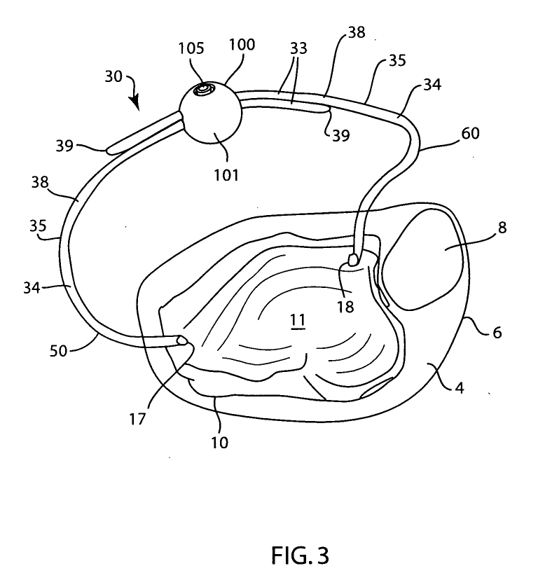

The human arm has a forearm 3, a wrist joint 4 and a hand 5 as shown in FIG. 1. The arm is formed by bones and soft tissues, such as muscle, tendons, veins, arteries, nerves, all of which are surrounded by a layer of skin 6 as shown in FIGS. 1 and 3. The two bones in the forearm 3 are the ulna 8 and the radius 10. Each bone 8 and 10 extend longitudinally along the length of the forearm 3 from the elbow (not shown) to the wrist 4, and is generally in side-by-side relation relative to the other bone. The ulna 8 is located along the outside or pinky finger side of the forearm 3. The radius 10 is located along the inside or thumb side of the forearm 3.

The radius 10 has a lower or distal end 11 proximal the wrist joint 4, and an upper end 12 proximal to the elbow. During childhood, the radius 10 grows outwardly from its ends 11 and 12, which include the metaphysis. This growth forms the elongated shaft 13 portion of the radius, which has a generally linear shape and longitudinal axis 14. The shaft 13 of the radius 10 is formed by an outer wall or cortex 15 that is generally hollow to define an intramedullary canal 16 that contains soft marrow (not shown). The tubular cortex 15 is relatively thick along the middle or central portion of the shaft 13, but thins towards its ends 11 and 12. The cortex 15 is particularly thin at its radial styloid process (RSP) 17 and dorso-ulnar cortex (DUC) 18 locations on its distal end 11 proximal the wrist 4.

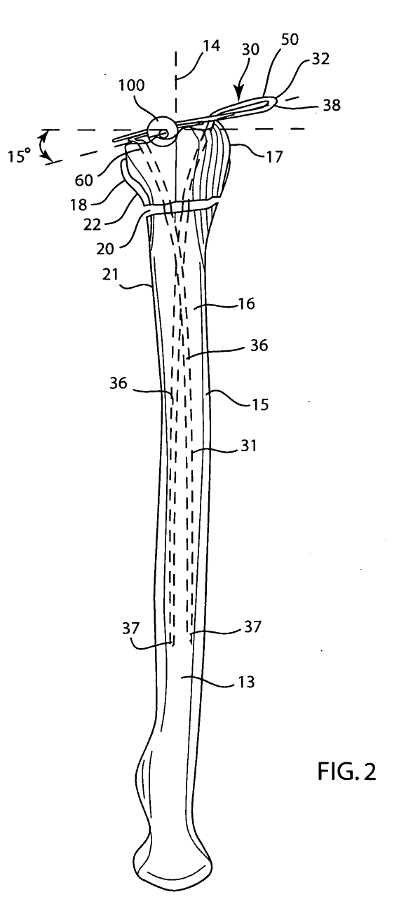

The distal ends of the ulna 8 and radius 10 combine to form a generally cup-shaped structure to accommodate the bone structure of the hand 5 and provide the angular mobility and strength of the wrist joint 4. When viewed from above as in FIG. 2, the surface forming the distal end 11 is angled about 15 degrees out of normal from the longitudinal axis 14 so that the thumb side extends further forward. Similarly, the top and bottom of the distal end 11 also extend further forward than its central portion. This gives the surface forming the distal end 11 a generally arcuate shape or partial cup-shaped structure that faces forward in general alignment with the longitudinal axis 14.

FIG. 1 shows a Colles' fracture of the radius 10. The fracture 20 breaks the radius 10 into two sections or pieces 21 and 22. The major piece 21 includes the bulk of the elongated shaft 14 and the upper or elbow end of the radius 10. The minor piece 22 includes the lower distal or wrist end 11 of the radius 10 and can include a portion of the shaft 14. During the healing process when the pieces 21 and 22 fuse back together, the minor piece 22 should be aligned with the longitudinal axis 14 of the radius 10 so that it once again forms a generally straight bone 10 with its cup shaped distal end 11 facing forward in its original direction in general alignment with longitudinal axis 14. Although the fracture 20 is shown as being a single relatively clean break extending generally normal to the longitudinal axis 14, it should be understood that the fracture could include multiple breaks and fragments, and can extend at an angle from side to side, from top to bottom, or both.

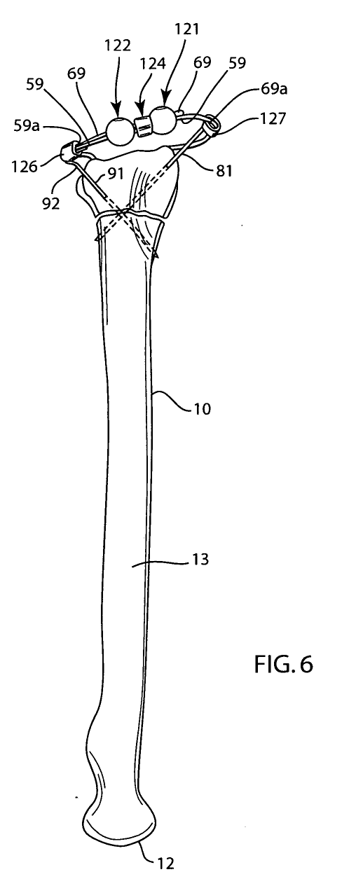

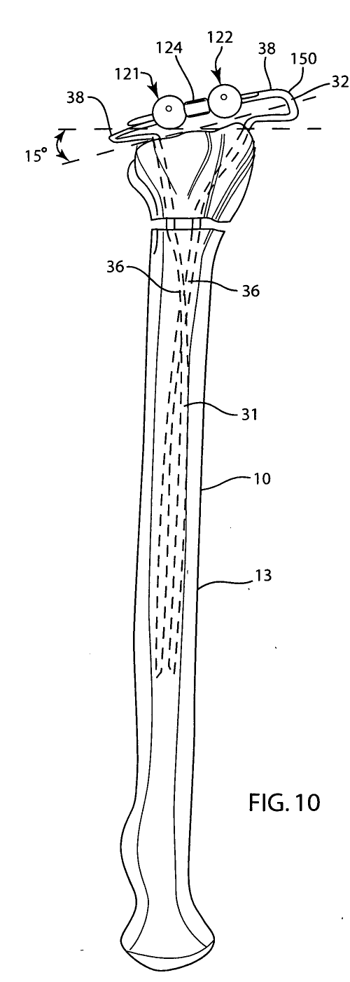

The present invention pertains to a fixator assembly for aligning pieces of a fractured bone having a hollow interior or internal canal and is generally depicted by Reference Number 30. The fixator 30 has an internal portion 31 that is surgically placed inside the bone and an external portion 32 that protrudes from the bone and skin 6. The fixator assembly 30 is formed by a set 33 of wires 34 and at least one fastener 44. In its basic form, the fixator assembly 30 includes two wires 34 joined by a single fastener 44 on its exterior portion 32 as shown in FIG. 1. For fractures 20 that are more difficult to stabilize, the fixator assembly 30 includes two additional wires 34 that are joined to the first set 33. A set 43 of fasteners 44 included securely joins the assembly 30 together as shown in FIG. 6. The exterior portion 32 is generally contoured to follow the natural curvature of the human wrist 4 so that it resembles a portion of a bracelet, but can have alternate shapes such as in FIG. 10, where the wires have right angles and linear segments to produce a more boxy shaped appearance. In each embodiment, the fixator assembly 30 aligns the pieces 21 and 22 of fractured bone 10, so that the bone is properly set to heal with the shorter piece 22 linearly or otherwise properly aligned in its original or desired relation relative to the other 21. The fixator 30 is particularly suited for setting a Colles' fracture 20 of the radius 10, and is intended for surgical placement by a trained surgeon.

Each wire 34 is made of stainless steel to help prevent corrosion and infection. Each wire 34 has a smooth exterior surface 35 and a diameter of about 1/16 inch (0.062 inch). This diameter gives the wires 34 a desired degree of rigidity to maintain the broken pieces 21 and 22 of bone 10 in alignment, and a desired degree of flexibility to facilitate their insertion into the bone, adjustment and joining of the wires 34, and secure attachment of the fasteners 44 as discussed below. Each wire 34 has a generally linear, elongated portion 36 that is placed in the pieces 21 and 22 of bone 10 and through the intramedullary canal 16. This elongated portion 36 has a sharp or ‘diamond’ tipped end 37 to allow the surgeon to use the wire 34 as a drill or auger to enter the bone 10 to create the desired opening in the cortex 15 and into the intramedullary canal 16 for inserting the wire. Each wire 34 also has a shaped portion 36 that extends from the bone 10 and through the skin 6. The shaped portion 38 remains entirely or predominantly external to the patient, and includes a lateral portion that generally extends laterally across the wrist 4. The lateral portion 38 has a blunted or rounded distal end 39 to avoid snags during use or puncturing the gloves of the surgeon.

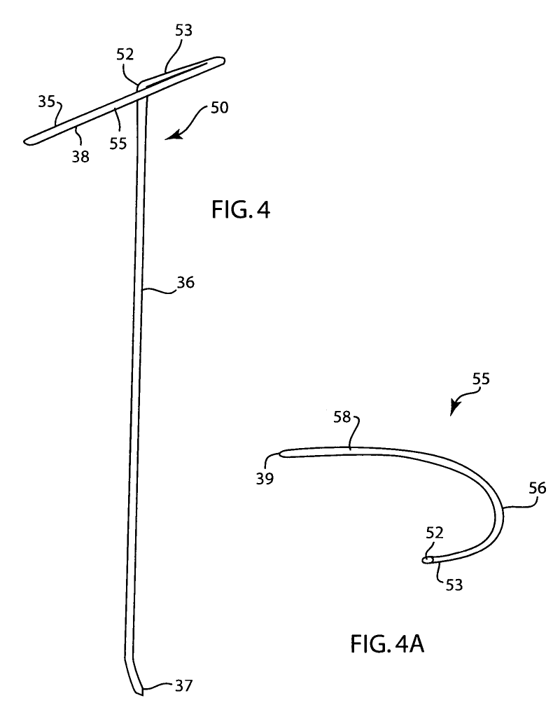

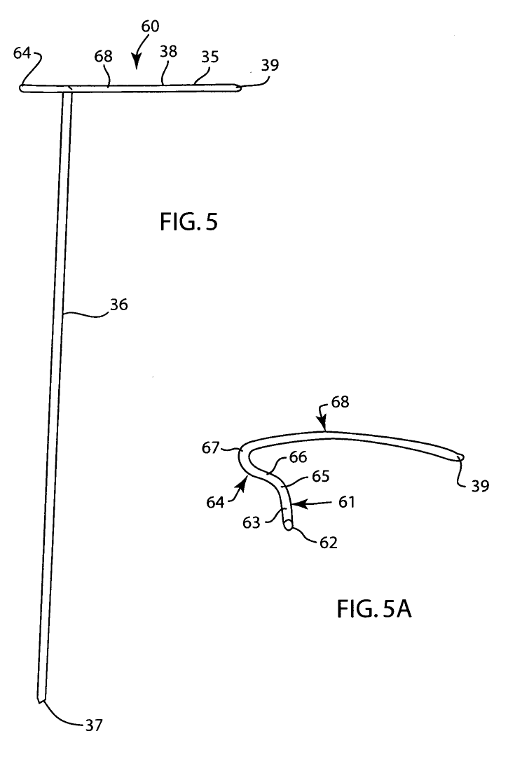

Each fixator assembly 30 has a set 33 of two primary positioning wires 50 and 60. The wire 50 and 60 are shown in their manufactured shape prior to surgical placement in FIGS. 4 and 5, respectively. Positioning wire 50 passes through the radial styloid process (RSP) 17 on the thumb side of the forearm 3, and is referred to as the RSP wire. Positioning wire 60 extends from the dorso-ulnar cortex (DUC) 18 on the ulnar side of the forearm 3 and is referred to as the DUC wire. The elongated portion 36 of each wire 50 and 60 has a length of about eight inches. Before surgical placement, the longitudinal portion 36 is substantially linear as shown in FIG. 4. The longitudinal portion 36 has a slight bend near its tip 37 for the purpose of ease of insertion as it follows the intramedullary canal.

The shaped portion 38 of RSP wire 50 has a radial portion 51 with a bend 52 and a linear segment 53 as best shown in FIG. 4A. The bend 52 forms an angle of about 75° relative to its elongated portion 36. When placed, segment 53 extends radially outward from the bone 10 toward the thumb side of the forearm 3, and through the skin 6. The radial segment 53 has a length of about ½ inch, and extends through and outward from the skin 6. The distance of outward extension from the skin 6 depends on the exact placement of the wire 50 through the radial styloid process 17 and the amount of soft tissue around the wrist 4. The ½ inch segment 53 provides the clearance to allow the wire 50 to be used for a wide variety of patients. The radial bend 52 and segment 53 form an abutment that engages the distal end 11 of radius 10 to longitudinally position the wire 60 relative to the radius 10.

RSP positioning wire 50 has a continuous bend 55 that extends from radial segment 53. This bend 55 has a generally curved shape that mimics the natural shape of the human wrist 4. The curve 55 extends in a continuously arcuate manner around the wrist 4, and includes a high curvature portion 56 and a low curvature portion 58. The high curvature portion 56 is located adjacent the radial portion 51 and has bend radius of about ½ inch. The low curvature portion 58 has a bend radius of about 1½ inches. The bend 55 extends upwardly to a point above the top of the wrist 4 and laterally across the wrist 4. The high curvature portion 56 provides most of the upward extension, and the low curvature portion 58 provides most of the lateral extension across the wrist 4. When placed in the intramedullary canal 16 and viewed form above as in FIG. 2, the low curvature portion 58 extends across the wrist 4 at an angle about 15° from normal relative to longitudinal axis 14, which is roughly equivalent to the 15° angle of the distal end 11 of the radius 10. The laterally extending, low curvature portion 58 includes a portion 59 that overlaps a corresponding portion of DUC wire 60 as in FIG. 1. The high curvature portion 56 includes a portion 59a that overlaps with one of the stabilizing wires when they are used as discussed below.

The shaped portion 38 of DUC wire 60 includes a radial portion 61 with a radial bend 62 and a radial segment 63 as best shown in FIG. 5A. The bend 62 forms an angle of about 90° relative to its linear, elongated portion 36. When placed, segment 63 extends radially outward from the bone 10 in an upward direction toward the top of the forearm 3. The radial segment 63 has a length of about ½ inch, and extends through and outward from the skin 6. The distance of outward extension from the skin 6 depends on the exact placement of the wire 50 through the dorso-ulnar cortex 18 and the amount of soft tissue around the wrist 4. The ½ inch segment 63 provides the clearance to allow the wire 50 to be used for a wide variety of patients. The radial bend 62 and segment 63 form an abutment that engages the distal end 11 of the radius 10 to longitudinally position the wire 60 relative to the radius 10.

DUC positioning wire 60 includes an offset portion 64 having an outward bend 65, offset segment 66 and inward bend 67. The offset portion 64 is desired because of the relatively short distance of about one inch between the radial styloid process 17 and dorso-ulnar cortex 18. The offset portion 64 provides additional lateral wire length for the overlapping union of wires 50 and 60 and insertion of fasteners 44 as discussed below. Immediately after the offset portion 64 is a continuous bend 68. This continuous bend 68 has a generally low curvature shape that is substantially the same as that of the low curvature portion 58 of wire 50. When placed in the forearm 3 and viewed form above as in FIG. 2, continuous bend 68 extends across the wrist 4 at an angle about 15° from normal relative to longitudinal axis 14, which is roughly equivalent to the 15° angle of the distal end 11 of the radius 10. Both curved portions 58 and 68 are generally concentric so they engage each other in flushly mating or continuous side-by-side relation. The laterally extending bend 68 includes a portion 69 that overlaps with overlap portion 59 of wire 50 as in FIG. 1. The bend 68 includes a portion 69a that overlaps a portion of one of the stabilizing wires when they are used as discussed below.

The fixator assembly 30 has an adjustable structure for placement on a variety of patients with a variety of wrist shapes and sizes. The curved portions 58 and 68 are substantially concentric when surgically placed and assembled. These curved portions 58 and 68 combine to form an effective diameter of said fixator assembly. The effective diameter determines the distance between the overlapping portions 59 and 69 and the skin surface 6 as in FIG. 3. Inward rotation of positioning wires 50 and 60 reduces the effective diameter of the fixator. The overlapping portions 59 and 69 increase in length and remain in substantially flush mating alignment. Outward rotation of positioning wires 50 and 60 increases the effective diameter of the fixator. The overlapping portions 59 and 69 decrees in length and remain in substantially flush mating alignment. A slight amount of flexing of the shaped portions 38 of the wires occurs with these adjustments.

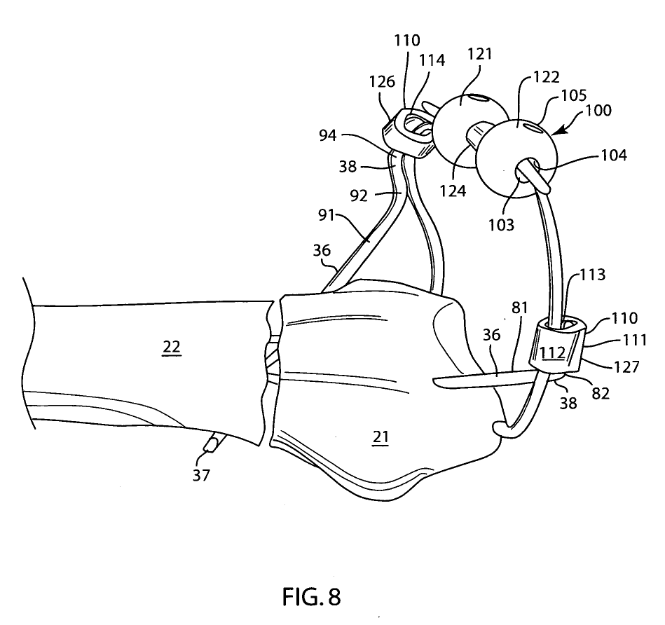

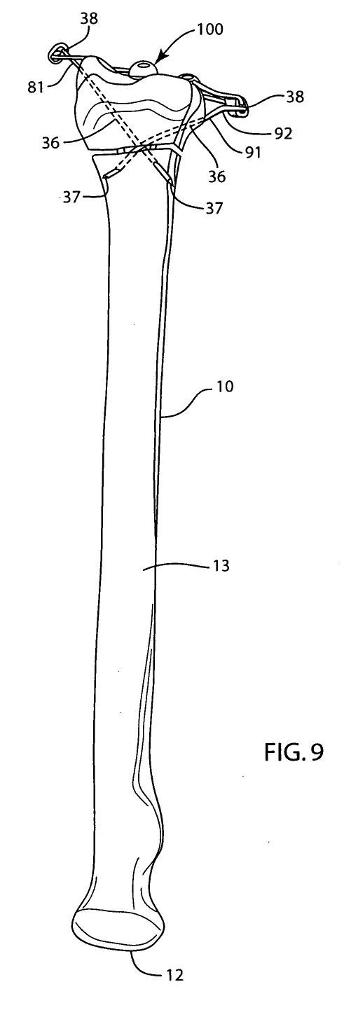

The set of wires 33 can include two or more interfragmentary wires 81 and 91 as shown in FIGS. 6-9. The elongated portion 36 of each interfragmentary wise 81 or 91 penetrates an upper portion of the tubular cortex 15 of the broken piece 22 proximal to the distal end 11 of the radius. Each wire 81 or 91 extends through the upper side of the fractured piece 22 at an angle such that the wire extends into the intramedullary canal 16, across the fracture 20, and into the opposite cortex 15 of the major portion 21 of the radius 10. Interfragmentary wire 81 penetrates the upper side of the cortex 15 proximal to the radial styloid process 17 and proceeds laterally at an angle of about a 45° towards the opposite or ulnar side of the radius bone 10, and longitudinally at an angle of about a 45° towards the opposite or ulnar surface of the radius bone 12. Similarly, interfragmentary wire 91 penetrates the upper surface of the radius bone cortex 15 proximal the dorso-ulnar cortex 18 and proceeds laterally at and angle of about 45° towards the opposite or radial side of the radius bone 10, and longitudinally at an angle of about 45° towards the opposite and proximal aspect of the radius bone 12.

Interfragmentary wire 81 has a shaped portion 38 with a first bend 82 and a short attachment segment 89 proximal its rounded distal end 39. The attachment segment 89 is aligned in generally parallel linear relation to and engagement with the high curvature portion 56 of wire 50. Segment 89 forms an overlapping portion that overlaps a portion 59a of the continuous bend 55. Cross wire 91 has a shaped portion 38 that includes an upward bend 92 followed by a lateral bend 94 and a short attachment segment 99 located proximal its rounded distal end 39. The attachment segment 99 is aligned in generally parallel linear relation to and engagement with the continuous bend 68 of wire 60. Segment 99 forms an overlapping portion that overlaps a portion 69a of the continuous bend 68. Each securement segment 89 and 99 has a length of about ¼ inch. Given their relatively short length, segments 89 and 99 can be linear or bent to match the curvature of their overlapping portion 59a or 69a of wires 50 and 60. Although interfragmentary wires 81 and 91 are shown and described as being at 45° angles and overlapping with wires 50 and 60 at particular attachment/overlap positions 59a and 69a on the high curvature portion 56 and attachment/overlap bend 68, respectively, it should be understood that the surgeon can alter this angle or these attachment/overlap positions. The overlap positions 59a and 69a can be located on the low curvature portion 58 or the offset portion 64, respectively.

A single pinball 100 is used to securely join positioning wires 50 and 60 to form the fixator assembly 30 shown in FIG. 1. A set of fasteners 43 including two pinballs 100 and three crimp sleeves 110 join the wires 50, 60, 81, and 91 together to form the fixator 30 in FIG. 6. Each pinball 100 has a unitary body 101 made of substantially rigid plastic, such as Delrin®. Each pinball 100 has an outer surface 102 and a central opening or channel 103 that extends completely through the pinball. This channel 103 is defined by an inner surface 104 of the pinball 100. The central opening 103 has a continuous uniform diameter that is slightly less than ⅛ inch, or slightly less than the diameter of two wires 50, 60, 81 and 91. This diameter allows two wires 50 and 60, 50 and 81, or 60 and 91 to be simultaneously received by the channel 103 in a snug manner that permits a sliding insertion or retraction of the wires.

Each pinball 100 has a threaded radial opening for receiving a setscrew 105. The threaded set screw 105 is operably movable from a release position where the screw does not extend into the central opening 103 to an engaged position where the tip of the screw enters the central opening and engages one or both of the wires passing through the pinball. Operable rotation of the set screw 105 into its engaged position against one or both of the wires 50 and 60 compresses the wires against each other and against the inner surface 104 of the ball 100 to securely fix the wires in place. Although the opening 103 is sized to receive two mating wires 50 and 60, 50 and 81, or 60 and 91, the operable extension of the set screw 105 enables the pinballs 100 to be secured to a single wire, such as to enclose the rounded ends 39 of the wires, or to decorate the fixator 30 as discussed below. The setscrews 105 allow the surgeon to readjust the fixator 30 and its alignment of the pieces 21 and 22 of the bone 10 after its initial placement during surgery, such as during a follow up visit a day or two after surgery.

Crimp sleeves 110 secure the wires 50, 60, 81 and 91 together in fixator assemblies 30 such as in FIG. 6. The crimp sleeves 110 are made of stainless steel or aluminum and have a length of about 1 1/2 the diameter of the pinball 100 and a width of about ½ the diameter of the pinball. The conventional crimp sleeves 110 are slid over two concentric or mating wires 50 and 60, 50 and 81, or 60 and 91, and crimped in a conventional manner to compress against and securely bind each set of mating wires together. Contrary to the pinballs 100, the crimp sleeves 110 are a more permanent fastener that are not intended to be loosened at a later date other than to remove the fixator 30 when the fracture 20 is healed. The pinballs 100 and crimp sleeves 110 are inserted onto the wires 34 after the wires have been surgically placed into the forearm 3 and radius 10 of the patient. The pinballs 100 and crimp sleeves 110 are free to slide along the length of one or two wires 34 prior to fasten them to the wires thus securely binding the wires together. The sliding aspect of the fasteners 44 allows the surgeon to align the wires 34 into their desired bone aligning positions before securing the wires via the fasteners 44. While the second embodiment shows wires 50 and 60 joined together by two pinballs 100 that straddle a central crimp sleeve 110, in some applications only one pinball 100 is required as previously discussed.

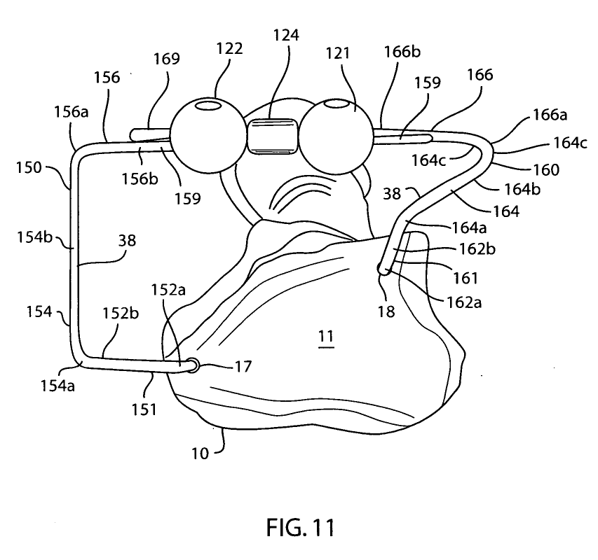

The lateral portion 38 of each wire 50 and 60 can have alternate shapes, such as in FIGS. 10 and 11. In this embodiment of the fixator 30, a modified RSP wire 150 has a lateral portion 38 with a radial portion 152 formed by a radial bend 152a and a radial segment 152b. The lateral portion 38 also includes an upward extending portion 154 formed by an upward bend 154a and an upward segment 154b. The lateral portion 38 further includes a crosswise portion 156 formed by a crosswise bend 156a and a crosswise segment 156b. Each bend 152a, 154a and 156a forms an angle of about 90°, and each segment 152b, 154b and 156b is substantially linear to give wire 150 a boxy or square shaped appearance.

This fixator 30 also has a modified DUC wire 160. The modified wire 160 has a lateral or external portion 38 with a radial portion 161 formed by a radial bend 162a and a radial segment 162b. The lateral portion 38 also has an offset portion 164 with an outward bend 164a, a linear segment 164b and an inward bend 164c. The radial portion 161 and offset portion 164 are substantially similar to the corresponding portions of wire 60 in the curved embodiment. However, the external portion 38 of the modified wire 160 has a crosswise portion 166 formed by a 90° crosswise bend 166a and a substantially linear crosswise segment 166b. The overlapping portions 159 and 169 are substantially linear for uniform engagement.

### Surgical Placement of Fixator

Although the process of placing the fixator in the forearm 3 and radius 10 of a patient should be apparent from the above, the following is provided to further assist the reader in understanding the placement process. Prior to surgery, the arm of the patient is secured into a desired position with top of the wrist 4 facing up and the hand 5 angled downward relative to the forearm 3 to facilitate access to the distal end 11 of the radius 10. The skin and tissues are spread to reveal the radial styloid process 17 and the dorso-ulnar cortex 18 of the radius 10.

The surgeon uses the sharp, diamond shaped tip 35 of the RSP wire 50 as an auger to gain entry to the medullary bone of the radial styloid process 17. The linear, elongated portion 34 of the wire 50 is then inserted into the intramedullary canal 16. The sharp, diamond shaped tip 37 of the DUC wire 60 is used as an auger to gain entry to the bone of the dorso-ulnar articular cortex 18. The linear, elongated portion 34 of the wire 60 is then inserted into the intramedullary canal 16. Because the radial styloid process 17 and the dorso-ulnar cortex 18 are both laterally offset from the center of the intramedullary canal 16, the wires 50 and 60 must be inserted at an angle into the canal. Although the wires 50 and 60 are biased to remain in their manufactured shapes, when their linear elongated portions 34 are inserted, they engage the opposite walls of the canal 16 and bend to give their elongated portions 34 a curved shape as seen in FIGS. 1 and 10. The stiffness of each wire 50 or 60 resists this bending and generates a force pushing against the tubular cortex 15 and the bone around the radial styloid process 17 or dorso-ulnar cortex 18 to help hold the wires in place.

The wires 50 and 60 are positioned so that their radial bends 52 and 62 abut the distal end 11 of the radius 10. The bends 52 and 62 are placed against the remaining portions of the radial styloid process 17 and dorso-ulnar cortex 18, respectively. This abutting engagement sets the longitude position of the wire 50 relative to the radius bone 10. When viewed from above as in FIGS. 2 and 10, the elongated portions 34 of the wires 50 and 60 are inserted into the radius 10 at an angle. The angle of insertion is about 15° out of parallel with the longitudinal axis 14 of the radius 10. Even though the RSP wire 50 is manufactured with its lateral portion 58 angled 30° out of normal to its longitudinal portion 34 (FIG. 4), the angled insertion of the wire results in its lateral portion being in generally parallel alignment with the angled the surface of the distal end 11 of the radius 10. Similarly, even though DUC wire 60 is manufactured with its lateral portion 68 normal to its elongated portion 34 (FIG. 5), the angled insertion of the wire results in the lateral portion being in generally parallel alignment with the angled surface of the distal end 11. Thus, the lateral potions 58 and 68 of the wires 50 and 60 are in generally side-by-side alignment when viewed from above without the need for additional bending or contorting of the wires. (FIGS. 2, 6 and 10). A portion 59 and 69 of each crosswire 58 and 68 overlaps the other.

The surgeon determines if only positioning wires 50 and 60 are required, or whether one, two or more stabilizing interfragmentary wires 81 and 91 are also required. When necessary, interfragmentary stabilizing wires 81 and 91 are placed to further join and align the fracture fragments 21 and 22 of bone 10. One hole is drilled at the desired angle as discussed above for inserting wire 81, and another hole is drilled at the desired angle for inserting wire 91. Each hole extends through the cortex 15 of each piece 21 and 22. Wire 81 is inserted into its respective hole until its lateral portion 89 is in side-by-side or overlapping alignment with the portion 59a of wire 50. Similarly, wire 91 is inserted into its respective hole until its lateral portion 99 is in side-by-side or overlapping alignment with the portion 69a of wire 60.

The pinballs 100 and crimp sleeves 110 are slid onto the shaped portions 36 of the wires 50 and 60. This can be done after both wires 50 and 60 have been inserted into the radius 10. The fasteners 100 and 110 are easily slid into place after rotating RSP wire 50 slightly counterclockwise and/or rotating DUC wire 60 slightly clockwise when viewed from the front as in FIG. 3, 7 or 11, so that their crosswise portions 58 and 68 rotate out of side by-side alignment. The wire 50 with the continuous bend 55 (FIG. 4A) is particularly suited for receiving fasteners 100 and 110, because they can be slid onto the high curvature portion 56, which is beyond the overlapping segment 59 of its low curvature portion 58.

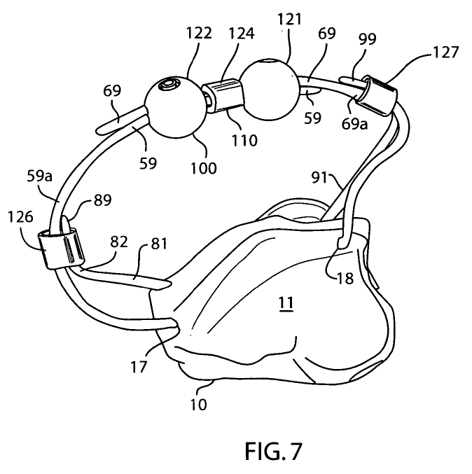

Once all the necessary and desired pinballs 100 and crimp sleeves 110 are slid or otherwise inserted onto the wires 50 and 60, the wire or wires are rotated back into side-by-side concentric or linear alignment with each other. The single pinball 100 (FIG. 3) or several pinballs and crimp sleeves 110 (FIG. 7 or 11) are then positioned over overlapping portions 59 and 69, 59a and 89 and 69a and 99. The pinballs 100 are then secured in place via the set screw 105, and the crimp sleeves 110 are secured in place via crimp deformation to rigidly secure or otherwise join the wires 50 and 60, 50 and 81 and wires 60 and 91 together, respectively. The crimp sleeves 110 can be left uncrimped during surgery until a follow up visit, so that the surgeon can ensure the proper alignment of the pieces 21 and 22 of the radius 10. If necessary, the surgeon can loosen the setscrews 105, readjust the wires 50 and 60, before crimping the sleeves 110 and rigidly securing the wires 50 and 60, 50 and 81, and 60 and 91 together.

The fixator assembly 30 is preferably provided as a kit. Each kit includes a set 33 of wires 34 and a set 43 of fasteners 44. The wires 44 include positioning wires 50 and 60, and interfragmentary wires 81 and 91. The fasteners 44 include a number of pinballs 100 and crimp sleeves 110. Each kit preferably includes two or more pinballs 100 and three or more crimp sleeves 110. Pinballs 100 in a specific kit have a specific color or a variety of colors, such as white, tan, red, blue yellow, green, violet, etc. Before surgery, the patient selects the color or colors of the pinballs 100 for their fixator 30. The patient also determines if any additional decorative pinballs 110 are to be installed on the fixator 30. As only one or two pinballs 100 are required to secure the wires 50 and 60 together and cover their rounded distal ends 39, additional pinballs are generally decorative in nature. The decorative pinballs 100 are positioned along the exterior portion 32 of the fixator 30, such as along the continuous bends 55 and 68 of wires 50 and 60. As noted above, pinballs 100 can be secured to two overlapping wires or a single wire. The surgeon uses the pinballs 100 with the selected color or colors to form the fixator 30.

While the invention has been described with reference to preferred embodiments, it will be understood by those skilled in the art that various changes may be made and equivalents may be substituted without departing from the broader aspects of the invention.

I claim:

1. A fixator assembly for aligning and setting a bone having and an internal canal, the bone being fractured into first and second pieces, and said fixator assembly comprising:
a first positioning wire having elongated and shaped portions, said elongated portion being adapted to extend substantially longitudinally through the canal of the first and second pieces of bone and through the fracture, said shaped portion being adapted to extend from the bone with a laterally extending portion extending across the bone in a crosswise direction;
a second positioning wire having elongated and shaped portions, said elongated portion being adapted to extend substantially longitudinally through the canal of the first and second pieces of bone and through the fracture, said shaped portion being adapted to extend from the bone with a laterally extending portion extending across the bone in an opposite crosswise direction, and each of said laterally extending portions have an overlapping portion that overlaps said other laterally extending portion, said overlapping portions being substantially coplanar and in substantially flush mating alignment when surgically placed and assembled; and,
a pinball having a unitary body with an external wall and an internal wall that defines a central opening extending completely through said pinball, said central opening being sized to simultaneously receive said overlapping portions, said pinball having a screw operably accessible from said external wall to selectively extend into said central opening and into engagement with at least one of said overlapping portions to compress them and fixedly join said positioning wires together.

2. The fixator assembly of claim 1, and wherein each of said laterally extending portions has a curved portion, said curved portion forming its said overlapping portion, and each of said curved portions has a similar curved shape to form said flush mating alignment.

3. The fixator assembly of claim 2, and wherein each of said curved portions has a radius of about 1.5 inches, and said wires are made of stainless steel and have a diameter of about 0.06 inches to give the wires a desired degree of flexability for surgical placement and assembly and a desired degree of stiffness to maintain the pieces of bone in alignment.

4. The fixator assembly of claim 2, and wherein said curved portions are substantially concentric when surgically placed and assembled, and said curved portions combine to form an effective diameter of said fixator assembly.

5. The fixator assembly of claim 4, and wherein the bone is a radius, the internal canal is an intramedullary canal and the fracture is a Colles fracture, the radius has a distal end with a radial styloid process and a dorso-ulnar cortex, said first positioning wire is adapted to extend through the radial styloid process, and said second positioning wire is adapted to extend through the dorso-ulnar cortex.

6. The fixator assembly of claim 5, and wherein said fixator assembly has an adjustable structure adapted for placement on a variety of patients with a variety of wrist shapes and sizes, each patient having a particular wrist with a particular shape and size; and,
wherein clockwise rotation of one of said positioning wires and counterclockwise rotation of an other of said positioning wires alters said effective diameter of said fixator assembly while maintaining said substantially flush mating alignment of said overlapping portions.

7. The fixator assembly of claim 1, and wherein said central opening of said pinball has a uniform diameter sized to snuggly receive said overlapping potions of said positioning wires.

8. The fixator assembly of claim 7, and wherein said positioning wires are made of stainless steel and have a uniform diameter of about 0.062 inches, and said central opening of said pinball has a diameter of slightly less than about 0.125 inches.

9. The fixator assembly of claim 5, and wherein the radius has a longitudinal axis and its distal end is generally 15 degrees out of normal to its longitudinal axis, and wherein said shaped portion of said first positioning wire has a continuous bend formed by a high curvature portion and a low curvature portion, said low curvature portion forming its said laterally extending portion, and wherein said laterally extending portions of said wires are generally parallel to the distal end of the radius when surgically placed and assembled.

10. A fixator assembly for aligning and setting a Colles fracture of a radius bone having a longitudinal axis, an intramedullary canal and a distal end with a radial styloid process and a dorso-ulnar cortex, the distal end being generally 15 degrees out of normal to the longitudinal axis, and the radius being fractured into first and second pieces, said first piece including the distal end of the radius, and said fixator assembly comprising:
a first positioning wire having elongated and shaped portions, said elongated portion being adapted to extend substantially longitudinally through the radial styloid process of the first piece, the intramedullary canal of the first and second pieces and the fracture, said shaped portion being adapted to extend from the radial styloid process with a laterally extending portion extending across the radius in a crosswise direction;
a second positioning wire having elongated and shaped portions, said elongated portion being adapted to extend substantially longitudinally through the dorso-ulnar cortex of the first piece, the intramedullary canal of the first and second pieces and the fracture, said shaped portion being adapted to extend from the dorso-ulnar cortex with a laterally extending portion extending across the radius in an opposite crosswise direction, and each of said laterally extending portions have an overlapping portion that overlaps said other laterally extending portion, said overlapping portions being substantially parallel to the distal end of the radius and in substantially flush mating alignment when surgically placed and assembled; and,
a fastener that defines a central opening extending completely through said fastener, said central opening being sized to simultaneously receive said overlapping portions, said fastener being operable to selectively compress said overlapping portions and fixedly join said positioning wires together.

11. The fixator assembly of claim 10, and wherein said fastener is a pinball having a unitary body with an external wall and an internal wall, said pinball having a securement device operably accessible from said external wall to selectively extend into said central opening and into engagement with at least one of said overlapping portions to compress said overlapping portions and fixedly join said positioning wires together.

12. The fixator assembly of claim 11, and wherein said overlapping portion is securely joined by two pinballs and a crimp sleeve.

13. The fixator assembly of claim 11, and wherein said fixator assembly further includes a stabilizing having elongated and shaped portions, said elongated portion being adapted to extend through the first piece and second pieces of bone, the intramedullary canal and the fracture, said shaped portion having an overlapping portion for flush mating alignment with one of said positioning wires when surgically placed and assembled, said overlapping portion being securely joined to said positioning wire by a fastener.

14. The fixator assembly of claim 10, and wherein each of said laterally extending portions has a curved portion, said curved portion forming its said overlapping portion, and each of said curved portions has a similar curved shape to form said flush mating alignment.

15. The fixator assembly of claim 14, and wherein each of said curved portions has a radius of about 1.5 inches.

16. The fixator assembly of claim 14, and wherein said curved portions are substantially concentric when surgically placed and assembled, and said curved portions combine to form an effective diameter of said fixator assembly.

17. The fixator assembly of claim 16, and wherein said fixator assembly has an adjustable structure adapted for placement on a variety of patients with a variety of wrist shapes and sizes, each patient having a particular wrist with a particular shape and size; and,
wherein clockwise rotation of one of said positioning wires and counterclockwise rotation of an other of said positioning wires alters said effective diameter of said fixator assembly while maintaining said substantially flush mating alignment of said overlapping portions

18. The fixator assembly of claim 10, and wherein said shaped portion of said first positioning wire has a high curvature portion and a low curvature portion.

19. The fixator assembly of claim 10, and wherein said bone is surrounded by a layer of skin, and each of said shaped portions of said positioning wires is adapted to extend through and is predominantly external the skin.

20. A fixator kit for aligning and setting a radius bone in a forearm with a layer of skin, the radius having an intramedullary canal and a Colles fracture dividing the bone into first and second pieces, said fixator kit comprising:
a plurality of wires, each of said wires having an elongated portion and a shaped portion with a laterally extending portion, said elongated portion being adapted to extend through the through the cortex of the first piece, intramedullary canal and fracture, said shaped portion being adapted to extend from the bone and through the skin, said laterally extending portions being substantially coplanar and in flush mating relation when surgically placed and assembled; and,
a plurality of fasteners including at least one pinball with a central opening and a cooperating set screw, said central opening being sized to simultaneously snugly receive two of said laterally extending portions, said set screw being operable to selectively extend into said central opening and engage and compress said laterally extending portions to fixedly join said wires together.

21. The fixator kit of claim 20 and wherein said fixator kit further includes a stabilizing having elongated and shaped portions, said elongated portion being adapted to extend through the first piece and second pieces of bone, the intramedullary canal and the fracture, said shaped portion having an overlapping portion for flush mating alignment with one of said positioning wires when surgically placed and assembled, said overlapping portion being securely joined to said positioning wire by a fastener.

22. The fixator kit of claim 20, and wherein said kit includes a variety of colored pinballs.
